(19) United States
(12) Patent Application Publication
(10) Pub. No.: US 2015/0241447 A1
(43) Pub. Date: Aug. 27, 2015

(54) VAGUS NERVE STIMULATION SCREENING TEST

(71) Applicants: Ralph J. Zitnik, Santa Barbara, CA (US); Jacob A. Levine, West Hempstead, NY (US)

(72) Inventors: Ralph J. Zitnik, Santa Barbara, CA (US); Jacob A. Levine, West Hempstead, NY (US)

(21) Appl. No.: 14/630,613

(22) Filed: Feb. 24, 2015

Related U.S. Application Data
(60) Provisional application No. 61/943,853, filed on Feb. 24, 2014.

(51) Int. Cl. G01N 33/68; A61B 5/00

(52) U.S. Cl. CPC G01N 33/6869; G01N 33/6863; A61B 5/0048; A61B 5/4029; G01N 2800/7095; G01N 2800/52; G01N 2333/525; G01N 2333/5412; G01N 2333/545

(57) ABSTRACT

A diagnostic screening test can be used to identify candidates for implantable vagus nerve stimulation devices. An analyte, such as a cytokine or inflammatory molecule, can be measured both before and after vagus nerve stimulation in order to determine the responsiveness of the patient. VNS can be delivered noninvasively or minimally invasively using short term electrical or mechanical stimulation.

### CROSS REFERENCE TO RELATED APPLICATIONS

This application claims priority to U.S. Provisional Application No. 61/943,853, filed Feb. 24, 2014, which is herein incorporated by reference in its entirety.

Some variations of the methods and apparatuses described in this patent application may be related to the following pending U.S. patent applications: U.S. patent application Ser. No. 12/620,413, filed on Nov. 17, 2009, titled “DEVICES AND METHODS FOR OPTIMIZING ELECTRODE PLACEMENT FOR ANTI-INFLAMMATORY STIMULATION,” now U.S. Pat. No. 8,412,338; U.S. patent application Ser. No. 12/874,171, filed on Sep. 1, 2010, titled “PRESCRIPTION PAD FOR TREATMENT OF INFLAMMATORY DISORDERS,” Publication No. US-2011-0054569-A1; U.S. patent application Ser. No. 12/917,197, filed on Nov. 1, 2010, titled “MODULATION OF THE CHOLINERGIC ANTI-INFLAMMATORY PATHWAY TO TREAT PAIN OR ADDICTION,” Publication No. US-2011-0106208-A1; U.S. patent application Ser. No. 12/978,250, filed on Dec. 23, 2010, titled “NEURAL STIMULATION DEVICES AND SYSTEMS FOR TREATMENT OF CHRONIC INFLAMMATION,” now U.S. Pat. No. 8,612,002; U.S. patent application Ser. No. 12/797,452, filed on Jun. 9, 2010, titled “NERVE CUFF WITH POCKET FOR LEADLESS STIMULATOR,” now U.S. Pat. No. 8,886,339; U.S. patent application Ser. No. 13/467,928, filed on May 9, 2012, titled “SINGLE-PULSE ACTIVATION OF THE CHOLINERGIC ANTI-INFLAMMATORY PATHWAY TO TREAT CHRONIC INFLAMMATION,” now U.S. Pat. No. 8,788,034; and U.S. patent application Ser. No. 13/338,185, filed on Dec. 27, 2011, titled “MODULATION OF SIRTUINS BY VAGUS NERVE STIMULATION,” Publication No. US-2013-0079834-A1. Each of these patent applications is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference. The following is a list of publications that are incorporated by reference:

Andersson U, Tracey K. Reflex principles of immunological homeostasis. Annu Rev Immunol 2012; 30:313.

Bruchfeld A, et al. Whole blood cytokine attenuation by cholinergic agonists ex vivo and relationship to vagus nerve activity in rheumatoid arthritis. J Int Med 2010; 268:94.

Dake M. Chronic cerebrospinal venous insufficiency and multiple sclerosis: Hostory and background. Techniques Vasc Intervent Radiol 2012; 15:94.

Ellrich J. Transcutaneous vagus nerve stimulation. Eur Neurological Rev 2011; 6:254-256.

Gao X, et al. Investigation of specificity of auricular acupuncture points in regulation of autonomic function in anesthetized rats. Autonomic Neurosc 1998; 88:109.

Huston J, et al. Transcutaneous vagus nerve stimulation reduces serum high mobility group box 1 levels and improves survival in murine sepsis. Crit Care Med 2007; 12:2762.

Koopman F, et al. Pilot study of stimulation of the cholinergic anti-inflammatory pathway with an implantable vagus nerve stimulation device in patients with rheumatoid arthritis. Arth Rheum 2012; 64 (10 suppl):S195.

M. L. Oshinsky, A. L. Murphy, H. Hekierski Jr., M. Cooper, B. J. Simon, Non-Invasive Vagus Nerve Stimulation as Treatment for Trigeminal Allodynia, PAIN (2014), doi: http://dx.doi.org/10.1016/j .pain.2014.02.009.

Peuker E. The nerve supply of the human auricle. Clin Anat 2002; 15:35.

Tekdemir I, et al. A clinico-anatomic study of the auricular branch of the vagus nerve and Arnold's ear-cough reflex. Surg Radiol Anat 1998; 20:253.

Yu L, et al. Low-level transcutaneous electrical stimulation of the auricular branch of the vagus nerve: A non-invasive approach to treat the initial phase of atrial fibrillation. Heart Rhythm 2013; 10:428.

Zhao Y, et al. Transcutaneous auricular vagus nerve stimulation protects endotoxemic rat from lipopolysaccharide-induced inflammation. Evid Based Complement Alternat Med. 2012;2012:627023. doi: 10.1155/2012/627023. Epub 2012 Dec. 29.

### FIELD

Embodiments of the invention relate generally to systems and methods for using vagus nerve stimulation for treatment, and more specifically to screening tests for identifying suitable patients for vagus nerve stimulation treatment.

### BACKGROUND

The vagus nerve mediates the inflammatory reflex, a mechanism the central nervous system utilizes to regulate innate and adaptive immunity (Andersson, 2012). The afferent arm of the reflex senses inflammation both peripherally and in the central nervous system, and down-regulates the inflammation via efferent neural outflow. The efferent arm of this reflex has been termed the “cholinergic anti-inflammatory pathway” (CAP). The reflex serves as a physiological regulator of inflammation by responding to environmental injury and pathogens with an appropriate degree of immune system activation (Andersson, 2012). CAP activation can also be harnessed to reduce pathological inflammation. Activating the CAP chronically using electrical neurostimulation of the vagus nerve is a feasible means of treating diseases characterized by excessive and dysregulated inflammation.

### SUMMARY OF THE DISCLOSURE

The present invention relates generally to systems and methods for using vagus nerve stimulation for treatment, and more specifically to screening tests for identifying suitable patients for vagus nerve stimulation treatment.

In some embodiments, a method for screening a patient for responsiveness to vagus nerve stimulation is provided. The method can include measuring a baseline level of an analyte; stimulating the vagus nerve after the step of measuring the baseline level of the analyte; measuring a post stimulation level of the analyte; comparing the post stimulation level of the analyte to the baseline level of the analyte; and determining whether the patient is a suitable candidate for an implantable vagus nerve stimulation device based on the step of comparing the post stimulation level of the analyte to the baseline level of the analyte.

In some embodiments, the step of stimulating the vagus nerve is done noninvasively.

In some embodiments, the step of stimulating the vagus nerve is done with transcutaneous electrical stimulation.

In some embodiments, the step of stimulating the vagus nerve is done with mechanical stimulation.

In some embodiments, the step of stimulating the vagus nerve comprises stimulating the auricular branch of the vagus nerve.

In some embodiments, the step of stimulating the vagus nerve comprises stimulating a cervical portion of the vagus nerve.

In some embodiments, the method further includes introducing an electrode intravascularly via percutaneous puncture; and positioning the electrode at a cervically located blood vessel proximate the vagus nerve.

In some embodiments, the method further includes introducing an electrode into the carotid sheath; and positioning the electrode within the carotid sheath such that the electrode is proximate the vagus nerve.

In some embodiments, the method further includes placing an electrode of a transcutaneous electrical nerve stimulation device on the patient's skin over the cervical vagus nerve.

In some embodiments, the method further includes introducing an expandable element intravascularly; positioning the expandable element within a cervically located blood vessel proximate the vagus nerve; and stimulating the vagus nerve by expanding the expandable element.

In some embodiments, the analyte is selected from the group consisting of cytokines and inflammatory mediators.

In some embodiments, the step of stimulating the vagus nerve comprises stimulating a cervical portion of the vagus nerve with a noninvasive form of stimulation selected from the group consisting of transcutaneous ultrasound energy, transcutaneous magnetic energy and transcutaneous RF energy.

In some embodiments, a method for screening a patient for responsiveness to vagus nerve stimulation is provided. The method can include collecting a first sample of cells from the patient; stimulating the vagus nerve after the step of collecting the first sample of cells from the patient; collecting a second sample of cells from the patient, the second sample collected after the step of stimulating the vagus nerve; inducing release of an analyte from the first sample and the second sample; comparing the release of the analyte from the first sample with the release of analyte from the second sample; and determining whether the patient is a suitable candidate for an implantable vagus nerve stimulation device based on the step of comparing the release of the analyte from the first sample with the release of analyte from the second sample.

In some embodiments, a method for screening a patient for responsiveness to vagus nerve stimulation is provided. The method can include measuring a baseline level of an analyte from a first sample obtained before vagus nerve stimulation; measuring a post stimulation level of the analyte from a second sample obtained after vagus nerve stimulation; comparing the post stimulation level of the analyte to the baseline level of the analyte; and determining whether the patient is a suitable candidate for an implantable vagus nerve stimulation device based on the step of comparing the post stimulation level of the analyte to the baseline level of the analyte.

In some embodiments, the analyte is selected from the group consisting of cytokines and inflammatory mediators.

In some embodiments, the patient is a suitable candidate for the implantable vagus nerve stimulation device when the post stimulation level of the analyte shows at least a predetermined reduction to the baseline level of the analyte. In some embodiments, the predetermined reduction is at least 10%, 20%, 30%, 40%, or 50%.

In some embodiments, the patient is a suitable candidate for the implantable vagus nerve stimulation device when the post stimulation level of the analyte shows at least a predetermined increase to the baseline level of the analyte. In some embodiments, the predetermined increase is at least 10%, 20%, 30%, 40%, or 50%.

In some embodiments, a method for screening a patient for responsiveness to vagus nerve stimulation is provided. The method can include inducing release of an analyte from a first sample and a second sample, wherein the first sample comprises cells taken from the patient before vagus nerve stimulation and the second sample comprises cells taken from the patient after vagus nerve stimulation; comparing the release of the analyte from the first sample with the release of analyte from the second sample; and determining whether the patient is a suitable candidate for an implantable vagus nerve stimulation device based on the step of comparing the release of the analyte from the first sample with the release of analyte from the second sample.

In some embodiments, the analyte is selected from the group consisting of cytokines and inflammatory mediators.

In some embodiments, the step of inducing release of the analyte comprises adding to the first sample and the second sample an inducing agent selected from the group consisting of bacterial lipopolysaccharide, Toll-like receptor activators, fragments of complement molecules, and activating antibodies directed against T cell or B cell surface receptors.

In some embodiments, the patient is a suitable candidate for the implantable vagus nerve stimulation device when the release of the analyte from the second sample shows at least a predetermined reduction to the release of the analyte from the first sample. In some embodiments, the predetermined reduction is 10%, 20%, 30%, 40%, or 50%.

In some embodiments, the patient is a suitable candidate for the implantable vagus nerve stimulation device when the release of the analyte from the second sample shows at least a predetermined increase to the release of the analyte from the first sample. In some embodiments, the predetermined increase is 10%, 20%, 30%, 40%, or 50%.

In some embodiments, the sample can be whole blood, serum, or supernatants from cultures of whole blood, or from cells isolated from whole blood.

In some embodiments, the analyte can be, for example, tumor necrosis factor (TNF), Interleukin (IL)-1 beta, or IL-6.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1C illustrate various embodiments of a mechanical stimulator.

FIG. 2 illustrates an embodiment of a transcutaneous electrical nerve stimulation device.

FIGS. 3A-3C illustrate various embodiments of minimally invasive electrical stimulation of the vagus nerve.

FIG. 3D illustrates an embodiment of minimally invasive mechanical stimulation of the vagus nerve.

### DETAILED DESCRIPTION

### Treatment of Rheumatoid Arthritis by Activating the CAP Using Electrically Active Implantable Medical Devices

A study of vagus nerve stimulation (VNS) using an electrically active surgically implanted medical device in 8 patients with active rheumatoid arthritis (RA) was performed at 4 investigative centers in Europe (Koopman, 2012). The device used an implanted helical coiled cuff lead to deliver electrical stimulation from an implanted pulse generator. After 6 weeks of stimulation, clinically significant improvements were seen in 6 of 8 patients as assessed by standard RA efficacy outcome measures such as the Disease Activity Score (DAS). These results provide proof-of-concept for the therapeutic use of VNS in RA as an alternative to small molecule and biological agent therapy. However, while the majority of the patients in the study responded, 2 of 8 were subjected to the risk and expense of surgical implantation of a device, yet did not have a meaningful clinical response. In order for VNS to be more successful as a therapy, it would be desirable to use a diagnostic screening test to preoperatively predict likelihood of clinical response to an implant, thus sparing patients who are unlikely to respond from undergoing an unnecessary surgical procedure. The diagnostic screening test may utilize various means, such as mechanical and electrical stimulation, to stimulate the vagus nerve noninvasively.

### CAP Activation by Stimulation of the Auricular Branch of the Vagus Nerve

Sensory fibers of the Auricular Branch of the Vagus Nerve (ABVN) innervate the skin of the cymba concha of the external ear. These fibers provide afferent input via the superior ganglion of the vagus to the brainstem nucleus of the solitary tract (NST). The neurons of the NST then project to efferent neurons originating in the dorsal nucleus and nucleus ambiguus of the vagus nerve, which then in turn project within the vagus nerve to the visceral organs (Ellrich, 2011 and references therein; Peuker, 2002).

This neuroanatomical pathway provides a potential mechanism for non-invasive activation of the CAP by induction of efferent vagal outflow through stimulation of the afferent ABVN pathway using mechanical or electrical stimulation of the skin of the cymba concha. The ABVN is particularly suitable for noninvasive stimulation because the nerves are located relatively close to the surface of the skin. Similarly, other portions of the vagus nerve that are similarly situated close to the patient's skin may be used for noninvasive stimulation. For example, the nerve may be located less than about 2, 1, 0.5 or 0.25 cm from the surface of the patient's skin. The terms “about”, “approximately” and the like can mean within 10%, 20%, or 30%.

The presence of such a functional reflex pathway in the ABVN may be demonstrated by the following observations: (1) the “Arnold's reflex” occurs in approximately 2% of the population and results in coughing or gagging in response to mechanical stimulation of the ear canal (Tekdemir, 1998); (2) ABVN electrical stimulation in rats resulted in parasympathetic vagally-mediated reductions in heart rate and blood pressure, and increased intra-gastric pressure. This response could be abrogated with atropine (Gao, 2008); (3) ABVN electrical stimulation in dogs was able to reverse the acute right atrial remodeling and reduction in threshold for inducible atrial fibrillation caused by rapid right atrial pacing. This protective effect of ABVN could be eliminated by bilateral transection of the vagus in its upper thoracic segment (Yu, 2013); (4) the ability of ABVN stimulation to activate the CAP and inhibit systemic inflammation in rodents was recently demonstrated (Zhao, 2012). In a rodent systemic endotoxemia model, transcutaneous electrical ABVN stimulation was compared to VNS delivered using a surgically placed cervical electrode. While cervical VNS was the most effective, ABVN stimulation caused significant reductions in circulating tumor necrosis factor (TNF), Interleukin (IL)-1 beta, and IL-6 (Zhao, 2012), thus demonstrating that the CAP can be effectively activated by transcutaneous auricular stimulation.

The CAP can be activated in the diagnostic screening test described herein by a short duration stimulation of the skin of the external ear, for example either mechanically or with an electric current. The stimulation duration can be between about 1 second to 24 hours and can be applied either continuously or intermittently throughout the duration. In some embodiments, the duration is less than about 1, 5 10, 20, 30, or 60 seconds. In some embodiments, the duration is less than about 1, 2, 3, 4, 5, 10, 15, 20, 30, 40, 50, or 60 minutes. In some embodiments, the duration is less than about 1, 2, 3, 4, 5, 6, 12, 18, or 24 hours.

The non-invasive stimulation may include mechanical stimulation of a body region such as the subject's ear. Examples of non-invasive stimulation devices are described in U.S. Publication No. 2008/0249439 to Tracey et al., which is herein incorporated by reference in its entirety. In particular, the cymba conchae region of their ear may be stimulated. The non-invasive stimulation may comprise mechanical stimulation between about 1 and 500 Hz, or 30 Hz and 500 Hz, or 50 Hz and 500 Hz. In some variations the stimulation is transcutaneous stimulation applied to the appropriate body region (e.g., the ear). For example, transcutaneous stimulation may be applied for an appropriate duration (e.g., less than 24 hours to less than 1 hour, less than 60 minutes to less than 1 minute, less than 60 seconds to less than 1 second, etc.), at an appropriate intensity and frequency. Stimulation that does not significantly affect cardiac measures may be particularly desirable, and the stimulation may be limited to such a range, or may be regulated by cardiac feedback (e.g., ECG, etc.).

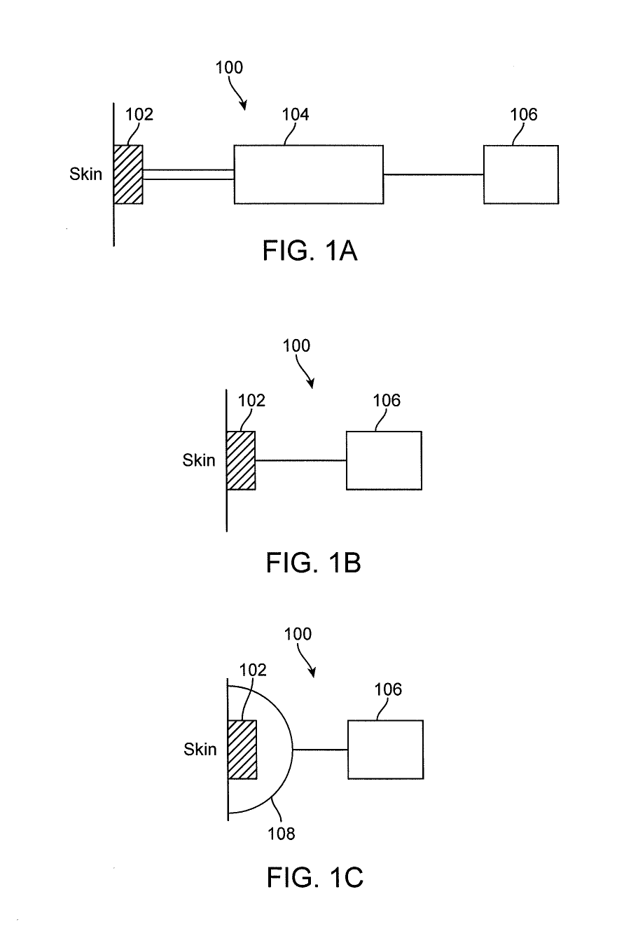

Also described herein are devices for non-invasively mechanically stimulating a subject's inflammatory reflex, as illustrated in FIGS. 1A-1C. These mechanical stimulation devices 100 may include an actuator 102, such as a movable distal tip region that is configured to mechanically stimulate at least a portion of a subject's ear, a handle 104, and a driver 106 configured to move the distal tip region between about 50 and 500 Hz. In some variations, the stimulation devices are part of a system including a stimulation device. In some embodiments, the actuator 102 can be directly adhered to the patient's skin, thereby removing the need for a handle. The actuator 102 can be coated with an adhesive or can be integrated into an adhesive pad or pod 108.

A stimulation device may include a controller configured to control the driver so that it applies stimulation within stimulation parameters. For example the controller (which may be part of the driver, or may be separate from the driver) may control the intensity (e.g., force, displacement, etc.), the timing and/or frequency (e.g., the frequency of repeated pulses during a stimulation period, the stimulation duration during the period of stimulation, the duration between stimulation periods, etc.), or the like. In some variations the controller is pre-programmed. In some variations, the controller receives input. The input may be control input (e.g., from a physician or the patient) that modifies the treatment. In some variations the stimulator device includes a therapy timer configured to limit the duration of stimulation. For example, the controller may be configured to limit the period of stimulation to less than 10 minutes, less than 5 minutes, less than 3 minutes, less than 1 minute, etc.

Any appropriate driver may be used. For example, the driver may be a motor, voice (or speaker) coil, electromagnet, bimorph, piezo crystal, electrostatic actuator, and/or rotating magnet or mass. For example, in some variations the driver is a mechanical driver that moves an actuator against the subject's skin. Thus, an actuator may be a distal tip region having a diameter of between about 35 mm and about 8 mm.

In some variation the stimulator includes a frequency generator that is in communication with the driver. Thus the driver may control the frequency generator to apply a particular predetermined frequency or range of frequencies to the actuator to non-invasively stimulate the subject.

The stimulator devices described herein may be hand-held or wearable. For example, also described herein are wearable device for non-invasively stimulating a subject's inflammatory reflex. These stimulator the devices may include an actuator configured to mechanically stimulate a subject's cymba conchae, a driver configured to move the distal tip region between about 1 and 500 Hz, or 30 Hz and 500 Hz, or 50 Hz and 500 Hz, and an ear attachment region configured to secure to at least a portion of a subject's ear.

Further description of a mechanical stimulator can be found in U.S. Publication No. 20080249439 to Tracey et al. which is herein incorporated by reference in its entirety for all purposes.

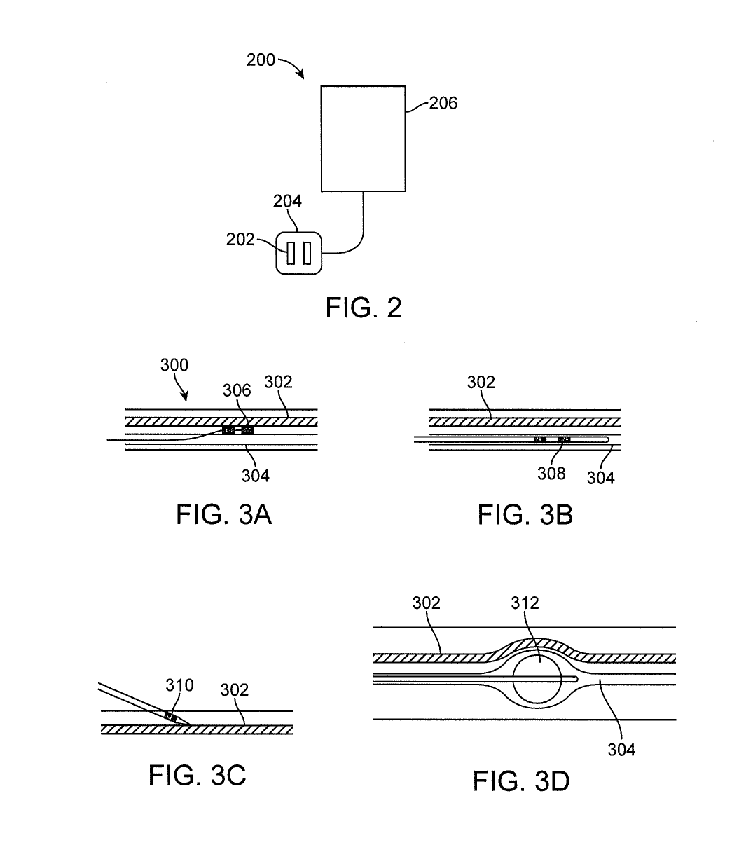

Transcutaneous electrical nerve stimulation (TENS) can be provided by an electrical stimulation device 200 having at least one electrode 202 that can be placed on the patient's skin, as illustrated in FIG. 2. The electrode 202 can be integrated into an adhesive patch or pad 204 that can be adhered to the patient's skin. A lead can connect the electrode with the housing 206 of the device. Alternatively, the housing can also be integrated with the adhesive patch or pad. A control or signal generator can deliver the electrical signal stimulus through the electrode. The signal amplitude can be between about 0.05 to 10 mA, or 0.05 to 15 mA, or 0.05 to 20 mA, or 0.05 to 25 mA, or 0.05 to 50 mA. The pulse width can be between about 100 and 1,000 μS. The pulse frequency can be between about 1 and 50 Hz, and the stimulus duration can be between about 1 second and 24 hours.

The electrical parameters can be similar when the electrical stimulation is provided by an electrode that has been inserted into the patient through minimally invasive techniques as described herein. For example, the signal amplitude can be between about 0.05 to 5 mA, or 0.05 to 10 mA, or 0.05 to 15 mA, 0.05 to 20 mA, or 0.05 to 25 mA or 0.05 to 50 mA.

### CAP Activation by Stimulation of the Cervical Vagus Nerve

Temporary electrical stimulation of the cervical vagus nerve for screening purposes can be performed using a variety of devices. For example, the device can include a non-cuffed lead 306 placed percutaneously through a blood vessel 304 such as the internal jugular vein on or near the vagus nerve 302 within the carotid sheath 300, as illustrated in FIG. 3A. Alternatively, as shown in FIG. 3B, a standard percutaneous catheter-directed intravascular electrode 308 can be positioned within the internal jugular 304 or other nearby blood vessel, in close anatomical apposition to the vagus nerve 302, with electrical stimulation delivered trans-vascularly. Other electrode or lead configurations, such as needle electrodes 310, can also be used to temporarily stimulate the vagus nerve 302, as shown in FIG. 3C.

Alternatively, direct mechanical stimulation of the cervical vagus nerve by physical manipulation has been demonstrated to activate the CAP in the rodent endotoxemia model (Huston, 2007). Angioplasty of the internal jugular vein is performed routinely using percutaneously inserted balloon-tipped angioplasty catheters (Dake, 2012). Transmural pressure can be exerted internally within the internal jugular vein 304 using such balloon catheters 312 in order to mechanically stimulate the nearby cervical vagus nerve 302 and activate the CAP, as shown in FIG. 3D. Other mechanical stimulators or expanders can also be used to provide mechanical stimulation

Alternatively, the cervical vagus nerve can also be stimulated using intravascular or transcutaneous ultrasound, transcutaneous magnetic energy, transcutaneous RF energy, and transcutaneous electric stimulation.

The CAP can be activated in the diagnostic screening test described herein by short duration (1 second to 24 hour) stimulation of the cervical vagus nerve using the above methods, either mechanically or with an electric current.

### Measurement of CAP Activation Using Bioassays Performed on Peripheral Blood Samples

The degree of CAP activation induced by the stimulation methods described above can be measured in a diagnostic screening test using several biological activity assays performed on either serum samples, supernatants from whole blood samples cultured in vitro in the presence of cytokine release stimulators, or supernatants from separated cellular constituents of whole blood cultured in vitro in the presence of cytokine release stimulators (Bruchfeld 2010). Other assays may be based on tissue samples or other biological fluids. The reduction or increase in these mediators that is observed between a pre-stimulus measurement and a post-stimulus measurement is indicative of the responsiveness of the patient to CAP activation, and may predict the response to a permanently implanted VNS system. For example, a predetermined change in level or concentration of a mediator, cytokine, or analyte from a baseline level or concentration before stimulation may indicate responsiveness of the CAP to stimulation and suitability of the patient for an implanted VNS system. Alternatively, the reduction or increase in inducible release of a cytokine, mediator, signaling molecule, or other analyte in a cell-based assay can also be used. In some embodiments, the predetermined change is a reduction of at least 10, 20, 30, 40 or 50%. In some embodiments, the predetermined change is an increase of at least 10, 20, 30, 40 or 50%.

### Diagnostic Screening Test

The diagnostic screening test can predict the likelihood of clinical response to the VNS implant prior to actual implantation surgery, for use in clinical decision-making regarding patient selection. Patients that are in need of or that may benefit from a VNS implant can be identified and given the diagnostic screening test. The test involves activating the cholinergic anti-inflammatory pathway for a short duration using techniques that are either non-invasive or minimally invasive as described herein. For example, the vagus nerve can be stimulated electrically or mechanically, as described herein. The extent of the patient's biological response to temporary CAP activation may then be assessed by measuring pathway-mediated inhibition of immune activation in assays performed on blood samples or other biological fluids taken before and after the stimulation. The extent of the patient's biological response to a brief non- or minimally invasive activation of the pathway can predict clinical response to the permanent implant.

Activation of the cholinergic anti-inflammatory pathway pathway may be achieved through any of the following stimulation techniques: (1) noninvasive transcutaneous stimulation of the auricular branch of the vagus nerve which innervates the skin of the cymba conchae of the ear, using either electrical or mechanical stimulation; (2) stimulation of the cervical vagus nerve using a catheter-directed temporary electrical lead/electrode, introduced intravascularly via percutaneous puncture, and positioned within the cervical internal jugular vein or other nearby vein under fluoroscopic or ultrasound guidance to place it in close apposition to the cervical portion of the vagus nerve; (3) stimulation of the cervical vagus nerve by a temporary catheter-directed non-cuffed electrical lead/electrode, introduced into the carotid sheath by percutaneous puncture, and positioned under fluoroscopic, ultrasound, and/or endoscopic guidance to place it in close apposition to the cervical portion of the vagus nerve within the carotid sheath; (4) stimulation of the cervical vagus nerve by trans-vascular mechanical pressure using an intravascular angioplasty balloon or other expandable element, introduced via percutaneous puncture and positioned under fluoroscopic or ultrasound guidance in close apposition to the cervical portion of the vagus nerve within the cervical internal jugular vein or other nearby vein; and (5) noninvasive transcutaneous cervical vagus nerve stimulation using a transcutaneous electrical nerve stimulation device placed over the vagus nerve on the skin of the patient's neck.

Assessment of the strength of CAP activation can measured using the change from pre- to post stimulation levels in any of the following parameters in a tissue sample or biological fluid such as whole blood, serum, or supernatants from cultures of whole blood, or from cells isolated from whole blood: (1) cytokines or inflammatory mediators or other analytes; and (2) reduction in inducible release of cytokines or other inflammatory mediators or other analytes, from in vitro culture of whole blood or isolated cells from blood. Such induction may be in the form of bacterial lipopolysaccharide, other Toll-like receptor activators, fragments of complement molecules (e.g., C5a), or activating antibodies directed against T cell or B cell surface receptors (e.g. anti CD3/anti CD28 antibodies).

### Alternative Stimulation Locations

Other regions of the subject's body may be alternatively or additionally stimulated, particularly regions enervated by nerves of the inflammatory reflex. For example, the non-invasive stimulation and other stimulation modalities described herein may be applied to the subject's area innervated by the seventh (facial) cranial nerve or cranial nerve V. The non-invasive stimulation and other stimulation modalities described herein may be applied to at least one location selected from: the subject's cymba conchae of the ear, or helix of the ear. In some variations, the non-invasive stimulation and other stimulation modalities described herein is applied to at least one point along the spleen meridian.

It is understood that this disclosure, in many respects, is only illustrative of the numerous alternative device embodiments of the present invention. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

What is claimed is:

1. A method for screening a patient for responsiveness to vagus nerve stimulation, the method comprising:
measuring a baseline level of an analyte;
stimulating the vagus nerve after the step of measuring the baseline level of the analyte;
measuring a post stimulation level of the analyte;
comparing the post stimulation level of the analyte to the baseline level of the analyte; and
determining whether the patient is a suitable candidate for an implantable vagus nerve stimulation device based on the step of comparing the post stimulation level of the analyte to the baseline level of the analyte.

2. The method of claim 1, wherein the step of stimulating the vagus nerve is done noninvasively.

3. The method of claim 2, wherein the step of stimulating the vagus nerve is done with transcutaneous electrical stimulation.

4. The method of claim 2, wherein the step of stimulating the vagus nerve is done with mechanical stimulation.

5. The method of claim 1, wherein the step of stimulating the vagus nerve comprises stimulating the auricular branch of the vagus nerve.

6. The method of claim 1, wherein the step of stimulating the vagus nerve comprises stimulating a cervical portion of the vagus nerve.

7. The method of claim 6, further comprising:
introducing an electrode intravascularly via percutaneous puncture; and
positioning the electrode at a cervically located blood vessel proximate the vagus nerve.

8. The method of claim 6, further comprising:
introducing an electrode into the carotid sheath; and
positioning the electrode within the carotid sheath such that the electrode is proximate the vagus nerve.

9. The method of claim 6, further comprising:
placing an electrode of a transcutaneous electrical nerve stimulation device on the patient's skin over the cervical vagus nerve.

10. The method of claim 1, further comprising:
introducing an expandable element intravascularly;
positioning the expandable element within a cervically located blood vessel proximate the vagus nerve; and
stimulating the vagus nerve by expanding the expandable element.

11. The method of claim 1, wherein the analyte is selected from the group consisting of cytokines and inflammatory mediators.

12. The method of claim 1, wherein the step of stimulating the vagus nerve comprises stimulating a cervical portion of the vagus nerve with a noninvasive form of stimulation selected from the group consisting of transcutaneous ultrasound energy, transcutaneous magnetic energy and transcutaneous RF energy.

13. A method for screening a patient for responsiveness to vagus nerve stimulation, the method comprising:
collecting a first sample of cells from the patient;
stimulating the vagus nerve after the step of collecting the first sample of cells from the patient;
collecting a second sample of cells from the patient, the second sample collected after the step of stimulating the vagus nerve;
inducing release of an analyte from the first sample and the second sample;
comparing the release of the analyte from the first sample with the release of analyte from the second sample; and
determining whether the patient is a suitable candidate for an implantable vagus nerve stimulation device based on the step of comparing the release of the analyte from the first sample with the release of analyte from the second sample.

14. A method for screening a patient for responsiveness to vagus nerve stimulation, the method comprising:
measuring a baseline level of an analyte from a first sample obtained before vagus nerve stimulation;
measuring a post stimulation level of the analyte from a second sample obtained after vagus nerve stimulation;
comparing the post stimulation level of the analyte to the baseline level of the analyte; and
determining whether the patient is a suitable candidate for an implantable vagus nerve stimulation device based on the step of comparing the post stimulation level of the analyte to the baseline level of the analyte.

15. The method of claim 14, wherein the analyte is selected from the group consisting of cytokines and inflammatory mediators.

16. The method of claim 14, wherein the patient is a suitable candidate for the implantable vagus nerve stimulation device when the post stimulation level of the analyte shows at least a predetermined reduction to the baseline level of the analyte.

17. The method of claim 16, wherein the predetermined reduction is at least 10%.

18. The method of claim 14, wherein the patient is a suitable candidate for the implantable vagus nerve stimulation device when the post stimulation level of the analyte shows at least a predetermined increase to the baseline level of the analyte.

19. The method of claim 18, wherein the predetermined increase is at least 10%.

20. A method for screening a patient for responsiveness to vagus nerve stimulation, the method comprising:
inducing release of an analyte from a first sample and a second sample, wherein the first sample comprises cells taken from the patient before vagus nerve stimulation and the second sample comprises cells taken from the patient after vagus nerve stimulation;
comparing the release of the analyte from the first sample with the release of analyte from the second sample; and
determining whether the patient is a suitable candidate for an implantable vagus nerve stimulation device based on the step of comparing the release of the analyte from the first sample with the release of analyte from the second sample.

21. The method of claim 20, wherein the analyte is selected from the group consisting of cytokines and inflammatory mediators.

22. The method of claim 20, wherein the step of inducing release of the analyte comprises adding to the first sample and the second sample an inducing agent selected from the group consisting of bacterial lipopolysaccharide, Toll-like receptor activators, fragments of complement molecules, and activating antibodies directed against T cell or B cell surface receptors.

23. The method of claim 20, wherein the patient is a suitable candidate for the implantable vagus nerve stimulation device when the release of the analyte from the second sample shows at least a predetermined reduction to the release of the analyte from the first sample.

24. The method of claim 23, wherein the predetermined reduction is at least 10%.

25. The method of claim 20, wherein the patient is a suitable candidate for the implantable vagus nerve stimulation device when the release of the analyte from the second sample shows at least a predetermined increase to the release of the analyte from the first sample.

26. The method of claim 25, wherein the predetermined increase is at least 10%.
